(19) United States
(12) Patent Application Publication
(10) Pub. No.: US 2020/0111569 A1
(43) Pub. Date: Apr. 9, 2020

(54) SYSTEM, COMPUTING DEVICE, AND METHOD FOR ANALYZING SLEEP-RELATED ACTIVITY PATTERN USING MULTIMODAL SENSOR DATA

(71) Applicant: ELECTRONICS AND TELECOMMUNICATIONS RESEARCH INSTITUTE, Daejeon (KR)

(72) Inventors: Ji Youn LIM, Daejeon (KR); Hyun Tae JEONG, Daejeon (KR); Ga Gue KIM, Daejeon (KR); Kyoung Ju NOH, Daejeon (KR); Seung Eun CHUNG, Daejeon (KR)

(21) Appl. No.: 16/552,864

(22) Filed: Aug. 27, 2019

(30) Foreign Application Priority Data
Oct. 4, 2018 (KR) 10-2018-0118322

(51) Int. Cl. G16H 40/67; G16H 50/20

(52) U.S. Cl. CPC G16H 40/67; G16H 50/20

(57) ABSTRACT

Provided are a system, computing device, and method for analyzing a sleep-related activity pattern using multimodal sensor data. The system includes a user device configured to be attached to a user's body and collect data through a sensor module and a computing device configured to recognize actions of the user from the data, generate action sequence sets on the basis of chronological order of the recognized actions of the user, cluster activities of the user as sleep activities and non-sleep activities on the basis of the action sequence sets, extract non-sleep activity patterns associated with the sleep activities through correlation analysis between sequences including time information of the clustered non-sleep activities and sleep activities, and provide the extracted non-sleep activity patterns to the user device.

### CROSS-REFERENCE TO RELATED APPLICATION

This application claims priority to and the benefit of Korean Patent Application No. 2018-0118322, filed on Oct. 4, 2018, the disclosure of which is incorporated herein by reference in its entirety.

### BACKGROUND

### 1. Field of the Invention

The present invention relates to a system, computing device, and method for analyzing a sleep-related activity pattern using multimodal sensor data.

### 2. Discussion of Related Art

As wearable devices are widely used, various services for sensing users' lifestyles are being provided, and services for analyzing sleep-related activities of people are also being proposed.

In other words, since it was disclosed that sufficient sleep affects not only people's health but also job performance, services for measuring a user's sleep status with a wearable device and assisting the user to manage a healthy life are being proposed.

However, these services provide only results of monitoring sleep and activity level and do not provide information about which habit related to sleep a user should change to improve the quality of his or her sleep.

It was disclosed that sleep hygiene is important for sound sleep, and based on this, cognitive-behavioral therapy for insomnia (CBT-I), sleep hygiene education (SHE), and mindfulness-based therapy are generally given to people with sleep disorders.

In general, international clinical guidelines recommend CBT-I as a first treatment, and CBT-I is basically composed of (1) sleep restriction for enforcing regular sleep time, (2) stimulus control for controlling stimuli which interrupt sleep, and (3) cognitive therapy for preventing worries about sleep or relevant actions. Various kinds of clinical research are being conducted on effects and methods of CBT-I, and clinical methods are being improved to minimize interference by external factors.

According to such a method, intervention of a counselor and the like is minimized so that a subject person may not be mentally stressed. In this case, however, the subject person should make an effort to keep a sleep diary and the like and check by himself or herself whether the effort was made.

Also, according to a related art, a user has the inconvenience of personally inputting physical information in addition to his or her sleep pattern and daily activity level. Further, since it is necessary to personally input activities known to affect sleep, it is difficult to receive accurate analysis results.

### SUMMARY OF THE INVENTION

The present invention is directed to providing a system, a computing device, and a method for analyzing a sleep-related activity pattern using multimodal sensor data, the system, computing device, and method automatically recognizing actions done in a user's daily life on the basis of on-body sensor data collected through a user device, classifying the actions into sleep activities and non-sleep activities, analyzing correlations therebetween, and providing action pattern information related to a lifestyle for sound sleep.

Objectives of the present invention are not limited to those mentioned above, and other objects which have not been mentioned above may be clearly understood by those of ordinary skill in the art from the following description.

According to an aspect of the present invention, there is provided a system for analyzing a sleep-related activity pattern using multimodal sensor data, the system including: a user device configured to be attached to a user's body and collect data through a sensor module; and a computing device configured to recognize actions of the user from the data, generate action sequence sets on the basis of chronological order of the recognized actions of the user, cluster activities of the user as sleep activities and non-sleep activities on the basis of the action sequence sets, extract non-sleep activity patterns associated with the sleep activities through correlation analysis between sequences including time information of the clustered non-sleep activities and sleep activities, and provide the extracted non-sleep activity pattern to the user device.

The user device may collect user data and environmental data as the data, and the computing device may recognize the user's actions on the basis of the user data and the environmental data.

As one action sequence set, the computing device may group an action started before a specific action is finished, an action started as soon as the specific action is finished, and an action which is started together with the specific action but finished at a different time among the recognized actions of the user.

The computing device may classify the action sequence sets on the basis of a time difference between the action sequence sets and the numbers of actions of the user included in the action sequence sets.

The computing device may generate a sequence of actions of the user having a correlation greater than or equal to a preset reference as one action sequence set.

The computing device may analyze the correlation on the basis of one or more of time information, location information, and exercise amount information of the user's actions.

The computing device may generate label information of the action sequence sets on the basis of domain knowledge about an activity categories based on time use survey and generate activities of the user by combining the action sequence sets into sequence patterns on the basis of time information included in the action sequence sets.

The computing device may statistically analyze frequencies of the sequence patterns of the action sequence sets on the basis of domain knowledge or statistical data about high-ranking items of a previously stored activity categories based on time use survey and generate the activities of the user by grouping the action sequence sets on the basis of results of the statistical analysis.

The computing device may extract characteristic information of the sleep activities on the basis of domain knowledge specialized in sleep, extract characteristic information of the non-sleep activities on the basis of the data received through the sensor module, and cluster the activities of the user on the basis of the extracted characteristic information.

The computing device may extract one or more of a sleep latency, an awake time during sleep, the number of times of awakening, and sleep efficiency as the characteristic information of the sleep activities and extract activity level information, information based on a heart rate, physical exercise information, and information on actions a certain time before sleep from the data received through the sensor module as the characteristic information of the non-sleep activities.

The computing device may perform a sequence analysis on clustered non-sleep activities which have a correlation of a preset value or more with the clustered sleep activities and extract sequence patterns of the clustered non-sleep activities.

The computing device may generate habit information to be provided to the user device on the basis of a frequently repeated sequence pattern among the sequence patterns of the clustered non-sleep activities.

The computing device may generate grade information of each of the non-sleep activity clusters and the sleep activity clusters by clustering the activities of the user and generate, when a non-sleep activity cluster associated with a first sleep activity cluster having a low grade in the grade information is recognized, a non-sleep activity cluster associated with a second sleep activity cluster having a higher grade than the first sleep activity cluster and occurring after the recognized non-sleep activity cluster as habit information to be provided to the user device.

The user device may further include a display module configured to output information generated or analyzed by the computing device.

According to another aspect of the present invention, there is provided a computing device for analyzing a sleep-related activity pattern using multimodal sensor data, the computing device including: a communication module configured to exchange data with a user device; a memory configured to store a program for analyzing a sleep-related activity pattern of a user on the basis of the data; and a processor configured to execute the program stored in the memory. When the processor executes the program and receives sensed data from the user device attached to the user's body through the communication module, the processor recognizes actions of the user from the data, generates action sequence sets on the basis of chronological order of the recognized actions of the user, clusters activities of the user as sleep activities and non-sleep activities on the basis of the action sequence sets, extracts non-sleep activity patterns associated with the sleep activities through correlation analysis between sequences including time information of the clustered non-sleep activities and the clustered sleep activities, and provides the extracted non-sleep activity patterns to the user device.

According to another aspect of the present invention, there is provided a method of analyzing a sleep-related activity pattern using multimodal sensor data, the method including: receiving data through a sensor module attached to a user's body; recognizing actions of the user from the data; generating action sequence sets on the basis of chronological order of the recognized actions of the user; clustering activities of the user as sleep activities and non-sleep activities on the basis of the action sequence sets; and extracting non-sleep activity patterns associated with the sleep activities through correlation analysis between sequences including time information of the clustered non-sleep activities and the clustered sleep activities.

The generating of the action sequence sets may include generating a sequence of actions of the user having a correlation greater than or equal to a preset reference as one action sequence set.

The clustering of the activities of the user as the sleep activities and the non-sleep activities on the basis of the action sequence sets may include: extracting characteristic information of the sleep activities on the basis of domain knowledge specialized in sleep; extracting characteristic information of the non-sleep activities on the basis of the data received through the sensor module; and clustering the activities of the user on the basis of the extracted characteristic information.

The method may further include generating habit information to be provided to a user device on the basis of a frequently repeated sequence pattern among sequence patterns of the non-sleep activity clusters.

The method may further include: generating grade information of each of the non-sleep activity clusters and the sleep activity clusters by clustering the activities of the user; and generating, when a non-sleep activity cluster associated with a first sleep activity cluster having a low grade in the grade information is recognized, a non-sleep activity cluster associated with a second sleep activity cluster having a higher grade than the first sleep activity cluster and occurring after the recognized non-sleep activity cluster as habit information to be provided to the user device.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a block diagram of a system for analyzing a sleep-related activity pattern according to an exemplary embodiment of the present invention;

FIG. 2 is a block diagram illustrating functionality of a computing device according to an exemplary embodiment of the present invention;

FIG. 3 is a diagram illustrating action sequence sets;

FIG. 4 is a diagram illustrating activities of a user whose action sequence sets are grouped;

FIG. 5 shows an example of non-sleep activity clusters which are associated with a sleep activity cluster and derived according to sequence analysis results;

FIG. 6 is a flowchart of a method of analyzing a sleep-related activity pattern according to an exemplary embodiment of the present invention;

FIG. 7A and FIG. 7B are flowchart illustrating a process of generating action sequence sets and a user's activities; and

FIG. 8 is a flowchart illustrating a process of generating a non-sleep activity pattern associated with a sleep activity cluster.

### DETAILED DESCRIPTION OF EXEMPLARY EMBODIMENTS

Hereinafter, exemplary embodiments of the present invention will be described in detail with reference to the accompanying drawings so that those of ordinary skill in the art of the present invention may readily implement the invention. However, the present invention may be implemented in a variety of different forms and is not limited to embodiments described herein. In the drawings, parts irrelevant to the description will be omitted so that the present invention may be clearly described.

Throughout the specification, when a part is referred to as “including” an element, the part does not exclude another element and may further include the other element unless particularly defined otherwise.

The present invention relates to a system 1, a computing device 200, and a method for analyzing a sleep-related activity pattern using multimodal sensor data.

The present invention includes various sensors and a display for a user interface. The present invention may acquire sleep and non-sleep activity data of an analyzable level through a user device 100 which may be worn by a user at all times and generate sleep-related habits and sleep-related activity patterns by analyzing the acquired data.

To this end, according to the present invention, (1) a user's low level actions are recognized from original data, and (2) action sequence sets including time information are made by considering chronological order of the recognized actions and correlations between the recognized actions and high level activities are defined from the action sequence sets. (3) The high level activities are classified into sleep activities and non-sleep activities which are not sleep activities, and clustering analysis is performed using characteristics based on sleep-related domain knowledge. After that, (4) correlations between sequences, which constitute sleep activity clusters and non-sleep activity clusters classified as analysis results and include time information, are analyzed, and activity patterns composed of non-seep activity clusters which affect sleep activity clusters are derived. Subsequently, activity patterns are analyzed according to the sleep activity clusters, and information which may be used in the user device 100 is generated and provided.

According to the present invention, a user's simple actions or activities are recognized and provided, and also information related to the user's lifestyle may be generated. Therefore, it is possible to analyze habit information of a person which affects a very important activity for health maintenance, that is, sound sleep, and to help the person to make a healthy lifestyle.

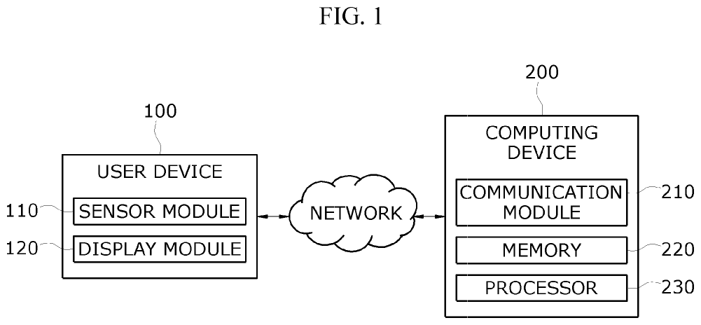

The system 1 for analyzing a sleep-related activity pattern using multimodal sensor data according to an exemplary embodiment of the present invention will be described below with reference to FIGS. 1 to 5.

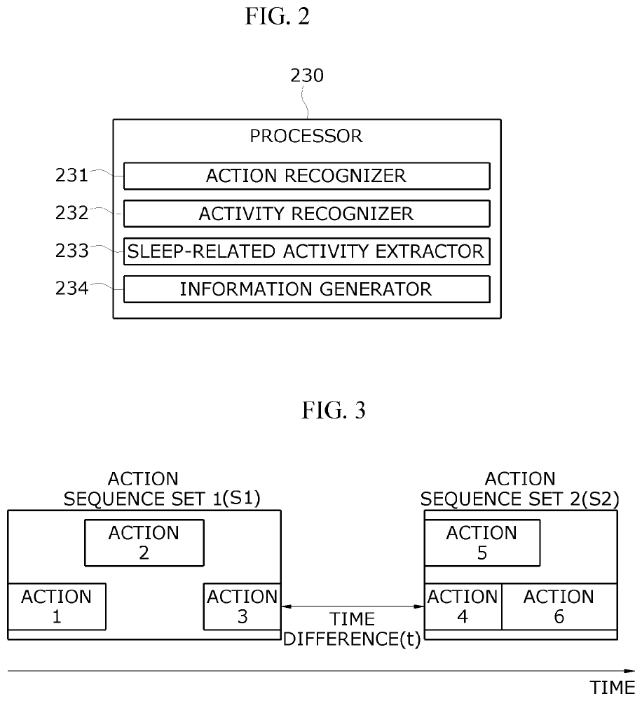

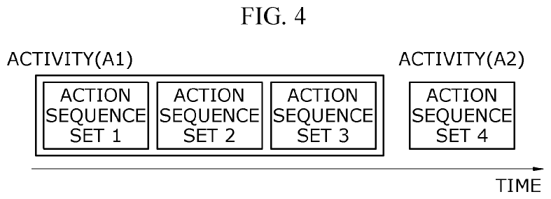

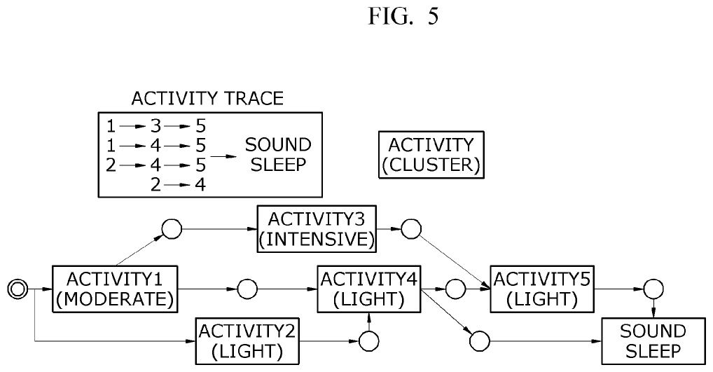

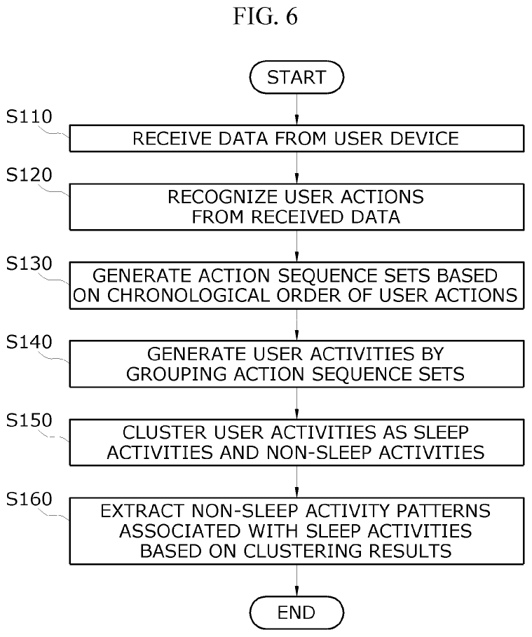

FIG. 1 is a block diagram of the system 1 for analyzing a sleep-related activity pattern according to an exemplary embodiment of the present invention. FIG. 2 is a block diagram illustrating functionality of the computing device 200 according to an exemplary embodiment of the present invention. FIG. 3 is a diagram illustrating action sequence sets. FIG. 4 is a diagram illustrating activities of a user whose action sequence sets are grouped. FIG. 5 shows an example of non-sleep activity clusters which are associated with a sleep activity cluster and derived according to results of analyzing correlations between sequences including time information.

Referring to FIG. 1 first, the system 1 for analyzing a sleep-related activity pattern according to an exemplary embodiment of the present invention includes the user device 100 and the computing device 200.

The user device 100 is attached to a user's body and collects user data and environmental data through a sensor module 110.

The user device 100 may be provided as a band type which is worn on a part of the user's body, such as a wrist or the head, or a patch type which is attached to a part of the body.

The sensor module 110 may include a sensor, such as an accelerometer, a geomagnetic sensor, a 9-axis gyro sensor, a heart rate sensor, a skin conductance sensor, and a skin temperature sensor, which may sense data related to the user's body or actions and a sensor which may sense data related to surroundings of the user, such as illumination, temperature, and global positioning system (GPS) and wireless fidelity (WiFi) signals.

The user device 100 may further include a display module 120 for outputting information generated or analyzed by the computing device 200 or providing a user interface related to a service screen for enabling the user to make an input or providing feedbacks related to user inputs and a lifestyle.

The computing device 200 is configured in the form of a server or a cloud. The computing device 200 recognizes actions and activities of the user and extracts and provides patterns of non-sleep activities associated with sleep activities to the user device 100. Also, the computing device 200 may process the extracted patterns of non-sleep activities and provide various feedback services for improving a lifestyle to the user so that the user may have a sound sleep.

The computing device 200 may include a communication module 210, a memory 220, and a processor 230.

The communication module 210 may exchange data with the user device 100. The communication module 210 may include a wireless communication module. The wireless communication module may be implemented using a wireless local area network (WLAN), Bluetooth, a high data rate (HDR) wireless personal area network (WPAN), Ultra-wideband (UWB), ZigBee, impulse radio, a 60 GHz WPAN, binary-code division multiple access (CDMA), WiFi, wireless universal serial bus (USB) technology, wireless high definition multimedia interface (HDMI) technology, and the like.

In addition, the communication module 210 may include all wired communication modules. The communication module 210 may be implemented as a power line communication device, a telephone line communication device, a cable home (multimedia over coax alliance (MoCA)), the Ethernet, Institute of Electrical and Electronic Engineers (IEEE) 1294, an integrated cable home network, and a recommended standard (RS)-485 control device. Accordingly, in an exemplary embodiment of the present invention, a wired communication module may be used to exchange data with the user device 100 at a high rate, and a port of a wired communication module (e.g., a micro USB port and a micro 5-pin terminal) may be used to provide a function of battery charging and the like.

In the memory 220, a program for analyzing sleep-related activity patterns of the user on the basis of data collected from the user device is stored, and the processor 230 executes the program stored in the memory 220. Here, the memory 220 collectively refers to a non-volatile storage device which maintains stored information even when power is not supplied and a volatile storage device.

For example, the memory 220 may include a NAND flash memory, such as a compact flash (CF) card, a secure digital (SD) card, a memory stick, a solid-state drive (SSD), and a micro SD card, a magnetic computer memory device, such as a hard disk drive (HDD), an optical disk drive, such as a compact disk read-only memory (CD-ROM) and a digital versatile disk (DVD)-ROM, and the like.

Detailed functions performed by the processor 230 of the computing device 200 will be described below with reference to FIG. 2.

The processor 230 generating lifestyle information which affects sleep includes an action recognizer 231, an activity recognizer 232, a sleep-related activity extractor 233, and an information generator 234.

The action recognizer 231 recognizes the user's actions through machine learning, such as deep learning, on the basis of the user data and the environmental data collected from the user device 100.

The user's actions may be simple actions such as walking, stopping, standing, sitting, lying, walking up the stairs, and walking down the stairs.

The activity recognizer 232 generates action sequence sets by analyzing sequences of the actions on the basis of recognition results of the action recognizer 231.

Specifically, as shown in FIG. 3, the activity recognizer 232 may group, as one action sequence, an action started before a specific action is finished, an action started as soon as the specific action is finished, and an action which is started together with the specific action but finished at a different time among the user's actions recognized by the action recognizer 231 on the basis of chronological order of the actions.

For example, in action sequence set 1 (S1) of FIG. 3, action 2 is started before action 1 is finished, and action 3 is started as soon as action 2 is finished. Therefore, action 1 to action 3 may be grouped as single action sequence set 1 (S1).

Also, in action sequence set 2 (S2), action 5 and action 6 are started at the same time but finished at different time points, and action 6 is started as soon as action 4 is finished and before action 5 is finished. Therefore, action 4 to action 6 may be grouped as single action sequence set 2 (S2).

Action sequences in such relationships may be classified on the basis of a case in which a time difference between grouped action sequences calculated considering time information is t seconds or more and a case in which the number of actions included in a grouped action sequence is limited to n.

Action sequences generated in this way may have different meanings but indicate similar actions. Therefore, a process of grouping similar action sequences as one action sequence set is performed thereafter.

To this end, the activity recognizer 232 analyzes similarities and correlations between the action sequences generated in a previous operation.

For example, a person's habit is not composed of identical movements during identical time periods. In other words, “stopping-walking-running-walking” and “walking-running-walking” may belong to two different jogging sets but may be “temporary running” for crossing a street on a green light.

Therefore, to determine whether target action sequences belong to the same set, the activity recognizer 232 performs correlation analysis. When a correlation is greater than or equal to k1 which is a preset reference, the activity recognizer 232 determines that the target action sequences have a meaningful relationship with each other and groups the target action sequences as one action sequence set. Otherwise, the activity recognizer 232 performs a process of analyzing the action sequences again.

The activity recognizer 232 may analyze correlations on the basis of one or more of time information, location information, and exercise amount information of the user's actions.

After action sequence sets are generated, the activity recognizer 232 gives a meaning to each action sequence set. At this time, the activity recognizer 232 may generate meaning label information for the action sequence sets on the basis of domain knowledge about an activity categories based on time use survey which is related to common living of people and built on the basis of, for example, statistics of the General Social Survey (GSS) of Statistics Canada. As an example, label information may be given as “meal,” “stroll,” “commute by car,” and the like.

Next, the activity recognizer 232 may generate activities of the user by combining the action sequence sets into sequence patterns on the basis of the time information included in the action sequence sets.

Although it is possible to detect a state of the user through an action sequence set to which a meaning has been given, sufficient information of the user's habits is not provided.

In other words, since a person's habit is expressed in a form, such as, “After lunch at the workplace, the user purchases a cup of coffee at a nearby café and takes a stroll with his or her coffee,” it is difficult to recognize the user's habit with a single action sequence set such as “lunch” or “stroll.”

Therefore, in order to detect the user's habits and generate habit information from the habits, a sequence pattern combination, such as “lunch”→“move by walking”→“sipping coffee” and “stroll,” should be made from several action sequence sets.

Such a sequence pattern combination may be made by generating data composed of rules, in which action sequence sets consecutively occur, with time information included in each action sequence set and then applying an algorithm for deriving correlation rules, such as the Apriori algorithm and the Tertius algorithm.

After sequence patterns of the action sequence sets are generated, the activity recognizer 232 statistically analyzes frequencies of the sequence patterns of the action sequence sets on the basis of domain knowledge or statistical data about high-ranking items of the previously stored activity categories based on time use survey. Then, the activity recognizer 232 may generate activities of the user by grouping the action sequence sets on the basis of results of the statistical analysis. For example, as shown in FIG. 4, the activity recognizer 232 may generate activity A1 of the user to include action sequence sets 1 to 3 and generate activity A2 to include action sequence set 4.

In other words, the activity recognizer 232 may analyze whether the action sequence sets are statistically similar to each other. For example, the activity recognizer 232 may analyze whether action sequence sets are statistically similar to each other on the basis of a frequency at which action sequence sets consecutively occur. When an analysis results indicates that a correlation is greater than or equal to k2 which is a preset reference, the activity recognizer 232 determines that the corresponding action sequence sets constitute a significant action sequence pattern and generates one user activity. Otherwise, the activity recognizer 232 analyzes action sequence sets again.

The user's activities defined in this way may be defined as “lunch at the workplace,” “commute,” “housework,” “leisure time,” and the like. The activity recognizer 232 may perform statistical analysis and generate user activities using domain knowledge which is transcendentally provided on the basis of statistical data or opinions of experts such as high-level classification items of an activity categories based on time use survey used in a national statistical office.

Next, the sleep-related activity extractor 233 clusters the user's activities as sleep activities and non-sleep activities on the basis of the action sequence sets generated by the activity recognizer 232 and extracts non-sleep activity sequence patterns associated with sleep activities through correlation analysis between sequences including time information of the clustered non-sleep activities and sleep activities.

Specifically, the sleep-related activity extractor 233 classifies the user's activities generated by the activity recognizer 232 into sleep activities and non-sleep activities and extracts characteristic information of each of the sleep activities and the non-sleep activities.

At this time, the sleep-related activity extractor 233 may extract characteristic information of the sleep activities derivable on the basis of domain knowledge specialized in sleep, for example, items which are generally used as references for diagnosis of sleep disorders in clinical psychology.

The characteristic information of the sleep activities may include one or more of a sleep latency, an awake time during sleep, the number of times of awakening, and sleep efficiency.

The sleep-related activity extractor 233 may extract characteristic information of the non-sleep activities on the basis of the user data and the environmental data received through the sensor module 110.

For example, characteristics which may be applied to daytime non-sleep activities associated with sleep may include one or more of activity level information obtained by calculating magnitudes of 3 axis values of the accelerometer and characteristics similar thereto, an inter-beat (RR) interval obtained from a heart rate, a rule-based physical exertion indicator which indicates whether a skin temperature and a skin conductance belong to normal ranges or abnormal ranges, and action information of a certain time (e.g., three hours) before sleep which is an important time period affecting sleep.

The sleep-related activity extractor 233 may cluster the sleep activities and the non-sleep activities on the basis of the characteristics. At this time, the sleep-related activity extractor 233 may apply various clustering algorithms according to the derived data characteristics.

When the clustering analysis is finished, the user's sleep activities and non-sleep activities are in result classified into an appropriate number of clusters.

For example, as clustering results, the sleep activities may be classified into three sleep activity clusters in which data constituting the activities recognized as “sleep” have grade information of “good sleep,” “moderate sleep,” and “poor sleep.” Among the non-sleep activities, a commute activity may be classified into three non-sleep activity clusters which have grade information of “long and arduous commute,” “moderate commute,” and “short and comfortable commute.”

Next, the sleep-related activity extractor 233 analyzes correlations between sequences including time information of non-sleep activity clusters having a correlation of a preset value or more with the sleep activity clusters and extracts sequence patterns of the non-sleep activity clusters.

In other words, the sleep-related activity extractor 233 may draw a causal relationship, a correlation, etc. by analyzing correlations between the sleep activity clusters and the non-sleep activity clusters generated as clustering results.

As examples of the results, it may be deduced that “good sleep” and “short and comfortable commute” have a high correlation and “poor sleep” and “long and arduous commute” have a high correlation.

In this case, the sleep-related activity extractor 233 performs sequence analysis to verify how frequently activities of non-sleep activity clusters having a correlation of a preset value k3 or more with the sleep activity clusters periodically occur during a data observation period.

The sleep-related activity extractor 233 may apply not only association rule analysis but also trend, seasonality, and cycle analysis as a sequence analysis method.

As a result of such a sequence analysis method, a sequence pattern composed of non-sleep activities, such as “long and arduous commute”—“work with little physical movement”—“dinner within three hours before sleep,” may be extracted.

To find non-sleep activity sequence patterns affecting the sleep activity clusters on the basis of the extracted sequence patterns, the sleep-related activity extractor 233 may derive significant non-sleep activity sequence patterns by applying process model analysis methodology.

Results drawn in this way may be expressed as shown in FIG. 5, and in practice, the same results may be drawn from all clusters related to sleep. For example, it is possible to derive a significant non-sleep activity sequence pattern in which a first action of a moderate level, a third action of an intensive level, and a fifth action of a light level are sequentially performed to lead to sound sleep.

When non-sleep activity sequence patterns for each sleep activity cluster are derived by the sleep-related activity extractor 233, the non-sleep activity sequence patterns are transferred to the information generator 234.

Then, the information generator 234 generates important habit information to be provided to the user device 100 from non-sleep activity sequence patterns related to each sleep activity cluster.

To this end, the information generator 234 may generate habit information to be provided to the user device 100 on the basis of a frequently repeated sequence pattern among non-sleep activity sequence patterns affecting each of the sleep activity clusters.

Also, when a non-sleep activity cluster associated with a first sleep activity cluster having a low grade is recognized, the information generator 234 may generate a non-sleep activity cluster associated with a second sleep activity cluster having a higher grade than the first sleep activity cluster and occurring after the recognized non-sleep activity cluster as the habit information to be provided to the user device 100 using grade information of the sleep activity clusters and the non-sleep activity clusters based on clustering results.

For example, the information generator 234 may generate information based on a non-sleep activity pattern and the like which is associated with a sound sleep cluster and capable of removing a non-sleep activity pattern related to a poor sleep cluster and occurring in the morning. In other words, when an “arduous and long commute” activity is recognized from a certain user and all non-sleep activity patterns including the “arduous and long commute” activity are associated with “poor sleep,” the information generator 234 may generate “slow lunch and then rest”→“static office work” associated with “sound sleep” as activity pattern information for the time after “arduous and long commute.”

For reference, elements shown in FIGS. 1 to 5 may be implemented in a software or hardware form such as a field programmable gate array (FPGA) or an application specific integrated circuit (ASIC) and may perform certain roles.

However, elements are not limited to software or hardware, and each element may be configured to be in an addressable storage medium or to execute one or more processors.

Therefore, by way of example, elements include elements, such as software elements, object-oriented software elements, class elements, and task elements, processes, functions, attributes, procedures, subroutines, segments of a program code, drivers, firmware, micro-codes, circuitry, data, databases, data structures, tables, arrays, and variables.

Elements and functions provided by the elements may be combined into a fewer number of elements or subdivided into additional elements.

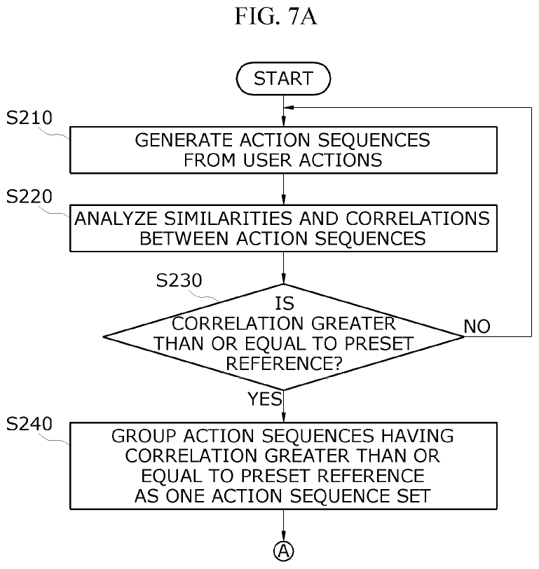

A method performed in the system 1 for analyzing a sleep-related activity pattern will be described below with reference to FIGS. 6 to 8.

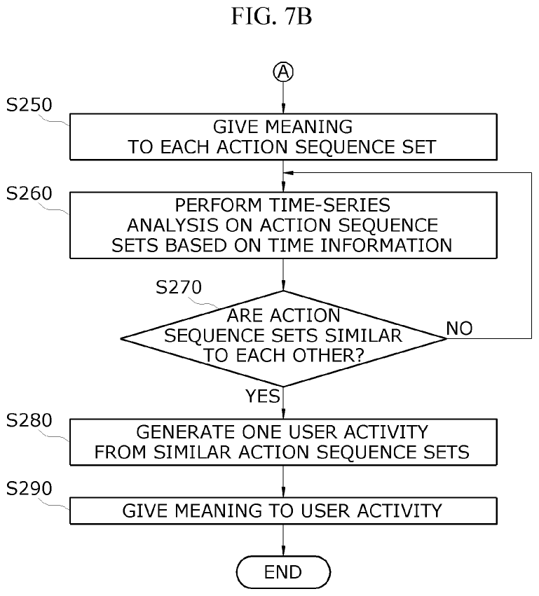

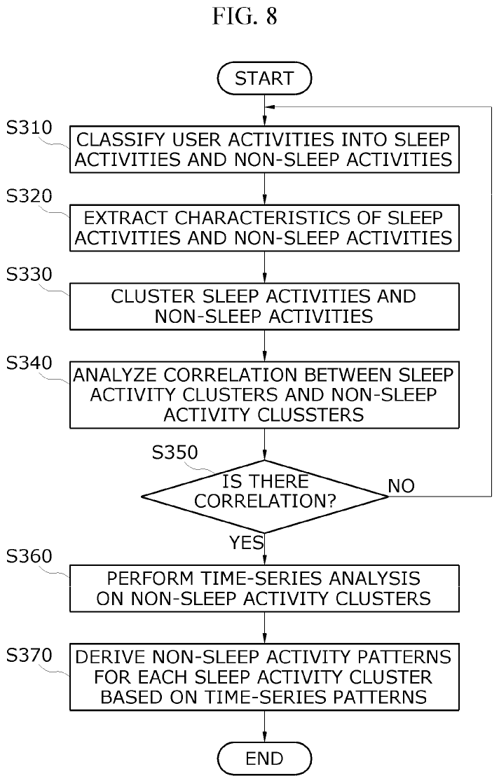

FIG. 6 is a flowchart of a method of analyzing a sleep-related activity pattern according to an exemplary embodiment of the present invention. FIG. 7A and FIG. 7B are flowchart illustrating a process of generating action sequence sets and a user's activities. FIG. 8 is a flowchart illustrating a process of generating a non-sleep activity pattern associated with a sleep activity cluster.

In a method of analyzing a sleep-related activity pattern according to an exemplary embodiment of the present invention, when data is received through the sensor module 110 attached to a user's body (S110), actions of the user are recognized from the data (S120).

Next, action sequence sets are generated on the basis of chronological order of the recognized actions of the user (S130), and activities of the user are generated by grouping the action sequence sets (S140).

Operation S130 and S140 are described in further detail with reference to FIG. 7A and FIG. 7B. First, action sequences are generated from the recognized actions of the user (S210), and similarities and correlations between the action sequences are analyzed (S220).

When an analysis result indicates that action sequences have a correlation greater than or equal to a preset reference (S230), one action sequence set is generated from the action sequences (S240), and a meaning is given to each action sequence set on the basis of domain knowledge (S250).

Next, correlation analysis is performed between sequences including time information of action sequence sets on the basis of time information included in action sequence sets to which meanings have been given (S260), and whether the action sequence sets are similar to each other is determined by statistically analyzing the analysis results (S270). When the action sequence sets are determined to be similar to each other, one user activity is generated from the action sequence sets (S280), and a meaning is given to the user activity on the basis of domain knowledge (S290).

Referring back to FIG. 6, user activities generated through the above operations are clustered as sleep activities and non-sleep activities (S150), and non-sleep activity sequence patterns associated with sleep activities are extracted through sequence analysis between the clustered non-sleep activities and the clustered sleep activities (S160).

Operations S150 and S160 are described in further detail with reference to FIG. 8. First, the user activities generated as groups of activity sequence sets are classified into sleep activities and non-sleep activities (S310), and characteristics are extracted from the activities (S320).

Next, the sleep activities and the non-sleep activities are clustered (S330), and correlations are analyzed between sleep activity clusters and non-sleep activity clusters which are clustering results (S340).

Then, sequence analysis is performed to verify how frequently activities of non-sleep activity clusters determined to have correlations with sleep activity clusters periodically occur during an observation period, and sequence patterns are generated (S360). On the basis of the sequence patterns, non-sleep activity sequence patterns are derived for each sleep activity cluster (S370).

Non-sleep activity patterns for each sleep activity cluster derived in this way may be provided to the user device 100.

Meanwhile, in the above description, operations S110 to S370 may be subdivided into additional operations or combined into a fewer number of operations according to an implementation example of the present invention. Also, some operations may be omitted as necessary, and the order of operations may be changed. Although omitted above, descriptions of FIGS. 1 to 5 may also be applied to the method of analyzing a sleep-related activity pattern illustrated with reference to FIGS. 6 to 8.

Existing service devices and technologies related to health trackers provide a service by recognizing a user's activities and analyzing a daily activity level or the user's living at a very macroscopic level such as home, commute, and workplace.

Even when a sleep is recognized, existing service devices and technologies provide only a sleep time versus a recommended sleep time and can neither analyze a pattern of changes in the user's sleep time, a relationship between a change in sleep and a lifestyle, and the like nor show the analysis results. Therefore, such a related technology or service may be a basis on which a user objectively determines a quality level of his or her sleep after looking at his or her sleep pattern but cannot provide any clue to important habits that he or she has not been able to recognize.

On the other hand, according to the above-described exemplary embodiment of the present invention, a high level activity pattern composed of simple actions is recognized through sequential hierarchy analysis, and information which may be directly applied to a service for changing a user's habits is generated and provided so that a user may recognize his or her habits. Therefore, a user may be aware of and change sleep-related habits that he or she is not able to recognize.

Meanwhile, an exemplary embodiment of the present invention may be implemented in the form of a recording medium including a computer program stored in a medium executed by a computer or instructions that can be executed by a computer. A computer-readable medium may be any available medium that can be accessed by a computer and includes all of volatile and non-volatile media and removable and non-removable media. The computer-readable medium may include both a computer storage medium and a communication medium. The computer storage medium includes all of volatile and non-volatile media and removable and non-removable media implemented with any method or technology, such as computer-readable instructions, data structures, program modules, or other data, for storing information. The communication medium includes computer-readable instructions, data structures, program modules, or other data in a modulated data signal, such as a carrier wave or other transport mechanism, and includes any information delivery medium.

Although the system 1 and method of the present invention have been described in association with specific exemplary embodiments, elements, some operations, or all operations thereof may be implemented using a computer system having general-use hardware architecture.

The foregoing description of the present invention is exemplary, and those of ordinary skill in the technical field of the present invention will appreciate that the present invention can be easily carried out in other detailed forms without departing from the technical spirit or essential characteristics of the present invention. Therefore, it should be noted that the above described embodiments are exemplary in all aspects and are not restrictive. For example, each element described to be a single type can be implemented in a distributed manner. Likewise, elements described to be distributed can be implemented in a combined manner.

The scope of the present invention is defined by the following claims rather than the above detailed description, and the meanings and ranges of the claims and all modifications derived from the concept of equivalents fall within the scope of the present invention.

What is claimed is:

1. A system for analyzing a sleep-related activity pattern using multimodal sensor data, the system comprising:
a user device configured to be attached to a user's body and collect data through a sensor module; and
a computing device configured to recognize actions of the user from the data, generate action sequence sets based on chronological order of the recognized actions of the user, cluster activities of the user as sleep activities and non-sleep activities based on the action sequence sets, extract non-sleep activity patterns associated with the sleep activities through correlation analysis between sequences including time information of the clustered non-sleep activities and sleep activities, and provide the extracted non-sleep activity patterns to the user device.

2. The system of claim 1, wherein the user device collects user data and environmental data as the data, and
the computing device recognizes the user's actions based on the user data and the environmental data.

3. The system of claim 1, wherein as one action sequence set, the computing device groups an action started before a specific action is finished, an action started as soon as the specific action is finished, and an action which is started together with the specific action but finished at a different time among the recognized actions of the user.

4. The system of claim 1, wherein the computing device classifies the action sequence sets based on a time difference between the action sequence sets and numbers of actions of the user included in the action sequence sets.

5. The system of claim 1, wherein the computing device generates a sequence of actions of the user having a correlation greater than or equal to a preset reference as one action sequence set.

6. The system of claim 5, wherein the computing device analyzes the correlation based on one or more of time information, location information, and exercise amount information of the user's actions.

7. The system of claim 1, wherein the computing device generates label information of the action sequence sets based on domain knowledge about an activity categories based on time use survey and generates activities of the user by combining the action sequence sets into sequence patterns based on time information included in the action sequence sets.

8. The system of claim 7, wherein the computing device statistically analyzes frequencies of the sequence patterns of the action sequence sets based on domain knowledge or statistical data about high-ranking items of a previously stored activity categories based on time use survey and generates the activities of the user by grouping the action sequence sets based on results of the statistical analysis.

9. The system of claim 1, wherein the computing device extracts characteristic information of the sleep activities based on domain knowledge specialized in sleep, extracts characteristic information of the non-sleep activities based on the data received through the sensor module, and clusters the activities of the user based on the extracted characteristic information.

10. The system of claim 9, wherein the computing device extracts one or more of a sleep latency, an awake time during sleep, a number of times of awakening, and sleep efficiency as the characteristic information of the sleep activities and extracts activity level information, information based on a heart rate, physical exercise information, and information on actions a certain time before sleep from the data received through the sensor module as the characteristic information of the non-sleep activities.

11. The system of claim 9, wherein the computing device performs a sequence analysis on clustered non-sleep activities which have a correlation of a preset value or more with the clustered sleep activities and extracts sequence patterns of the clustered non-sleep activities.

12. The system of claim 11, wherein the computing device generates habit information to be provided to the user device based on a frequently repeated sequence pattern among the sequence patterns of the clustered non-sleep activities.

13. The system of claim 11, wherein the computing device generates grade information of each of the non-sleep activity clusters and the sleep activity clusters by clustering the activities of the user and generates, when a non-sleep activity cluster associated with a first sleep activity cluster having a low grade in the grade information is recognized, a non-sleep activity cluster associated with a second sleep activity cluster having a higher grade than the first sleep activity cluster and occurring after the recognized non-sleep activity cluster as habit information to be provided to the user device.

14. The system of claim 1, wherein the user device further includes a display module configured to output information generated or analyzed by the computing device.

15. A computing device for analyzing a sleep-related activity pattern using multimodal sensor data, the computing device comprising:
a communication module configured to exchange data with a user device;
a memory configured to store a program for analyzing a sleep-related activity pattern of a user based on the data; and
a processor configured to execute the program stored in the memory,
wherein when the processor executes the program and receives sensed data from the user device attached to the user's body through the communication module, the processor recognizes actions of the user from the data, generates action sequence sets based on chronological order of the recognized actions of the user, clusters activities of the user as sleep activities and non-sleep activities based on the action sequence sets, extracts non-sleep activity patterns associated with the sleep activities through correlation analysis between sequences including time information of the clustered non-sleep activities and the clustered sleep activities, and provides the extracted patterns to the user device.

16. A method of analyzing a sleep-related activity pattern using multimodal sensor data, the method comprising:
receiving data through a sensor module attached to a user's body;
recognizing actions of the user from the data;
generating action sequence sets based on chronological order of the recognized actions of the user;
clustering activities of the user as sleep activities and non-sleep activities based on the action sequence sets; and
extracting non-sleep activity patterns associated with the sleep activities through correlation analysis between sequences including time information of the clustered non-sleep activities and the clustered sleep activities.

17. The method of claim 16, wherein the generating of the action sequence sets comprises generating a sequence of actions of the user having a correlation greater than or equal to a preset reference as one action sequence set.

18. The method of claim 16, wherein the clustering of the activities of the user as the sleep activities and the non-sleep activities based on the action sequence sets comprises:
extracting characteristic information of the sleep activities based on domain knowledge specialized in sleep;
extracting characteristic information of the non-sleep activities based on the data received through the sensor module; and
clustering the activities of the user based on the extracted characteristic information.

19. The method of claim 16, further comprising generating habit information to be provided to a user device based on a frequently repeated sequence pattern among sequence patterns of non-sleep activity clusters.

20. The method of claim 16, further comprising:
generating grade information of each of non-sleep activity clusters and sleep activity clusters by clustering the activities of the user; and
generating, when a non-sleep activity cluster associated with a first sleep activity cluster having a low grade in the grade information is recognized, a non-sleep activity cluster associated with a second sleep activity cluster having a higher grade than the first sleep activity cluster and occurring after the recognized non-sleep activity cluster as habit information to be provided to the user device.
